(19) United States
(12) Patent Application Publication
(10) Pub. No.: US 2005/0004483 A1
(43) Pub. Date: Jan. 6, 2005

(54) Bi-point detection type heart-rate monitor and its heart-rate monitoring method

(75) Inventor: Abdada Lin, Jhushan Township (TW)

Correspondence Address: BROWDY AND NEIMARK, P.L.L.C.;624 NINTH STREET, NW, SUITE 300, WASHINGTON, DC, 20001-5303, US

(73) Assignee: CHANG YOW INDUSTRY CO., LTD., Changhua Hsien (TW)

(21) Appl. No.: 10/661,514

(22) Filed: Sep. 15, 2003

(30) Foreign Application Priority Data
Jul. 3, 2003 (TW) 92118240

(51) Int. Cl. A61B 5/0402

(52) U.S. Cl. 600/509

(57) ABSTRACT

A bi-point detection type heart-rate monitor is constructed to include two electrically conductive contact terminals for the touching of the two hands or feet of a person, an amplifier-filter circuit adapted to sample impedance signal between the contact terminals, to amplify the impulse of the sampled impedance signal, and to remove noises from the sampled impedance signal, a waveform converter adapted to rectify outputted waveform from the amplifier-filter circuit into a square wave, and a processing and output circuit adapted to receive and process the square wave signal outputted by the wave form converter, to obtain the mean frequency of peaks of the received square wave signal by means of a computing process, and to output the frequency value thus obtained to a display unit for display.

### BACKGROUND OF THE INVENTION

1. Field of the Invention

The present invention relates generally to a heart-rate monitor adapted to monitor the frequency of a beating heart and, more particularly, to a bi-point detection type heart-rate monitor. The invention relates also to the heart-rate monitoring method used in the monitor.

2. Description of the Related Art

Heart-rate monitoring technology and apparatus have been well developed in medical care industry. In recent years, heart-rate monitoring devices have been intensively used in exercising machines, recreational apparatus, home apparatus, as well as personal apparatus. For example, advanced treadmills and standing bicycles have their handlebars provided with sensor means that detect the frequency of the beating of the user's heart. Therefore, the user can monitor one's heart-rate during exercise, knowing one's physical status. There are known weight scales having heart beat sensor means. When measuring the body weight, the weight scale indicates the heart-rate of the user.

According to conventional heart-rate monitoring methods, the human body is regarded as a bioelectrical resistor having a constant impedance, and an impulse is produced corresponding to each beating action of the heart, and therefore the heart-rate is measured. In a conventional heart-rate monitor, a proper voltage is applied to two parts of the body (normally the two hands or legs), and then the frequency of the impulse signal (indicative of the heart-rate) is measured after amplification and comparison of the voltage signals passing through the two parts of the body. According to the aforesaid prior art design, there are four contact terminals (four-point contact), and two hands (or legs) need to touch two contact terminals during examination. U.S. Pat. No. 5,337,753 is a typical design of the application of this technique. When this designed employed to an exercising machine, the two pairs of contact terminals are respectively located on the left and right ends of the handlebar for the touching of the hands. When used in a weight scale, the left half and right half of the footboard are respectively provided with two contact terminals for the contact of the left foot or right foot.

During the use of the aforesaid four-point detection type heart-rate monitor, the left hand and right hand (left foot and right foot) must simultaneously touch the corresponding two contact terminals so that the circuit can accurately measure the user's voltage signal. Because each hand (leg) of the user must simultaneously touch two contact terminals, the user must apply much effort to force the hands (legs) to force the non-planar palm surface of each hand (the non-planar surface of the bottom of the foot) into positive contact with the corresponding contact terminals. In order to prevent this problem, the handhold portions must be orthopedically engineered to fit the curvature of the palm surface or the surface of the bottom of the foot of a person. However, this design does not fit people of different body sizes (adults and children).

Further, when one hand (or leg) of the user does not touch the corresponding two contact terminals, for example, when the user loosening the muscles of one hand, causing the hand to touch one contact terminal only, at this time, the air between the hand and the other contact terminal is measured to obtain an impedance, producing a false value. Due to the interference of such a false value, an inaccurate heart-rate is produced, as shown in FIG. 1, causing the circuit to make an erroneous judgment.

### SUMMARY OF THE INVENTION

It is the main object of the present invention to provide a bi-point detection type heart-rate monitor, which uses two contact terminals for the touching of the user's two hands or two legs to obtain a single signal for heart-rate measuring without through a dual-signal comparison procedure as seen in prior art designs. Because each hand or leg needs only to touch one single terminal, the problem of contact error is eliminated.

It is another object of the present invention to provide a bi-point detection type heart-rate monitor, which uses a detection unit to detect positive contact between the user's hands or legs and the two contact terminals, preventing the production of a false value.

To achieve these objects of the present invention, the bi-point detection type heart-rate monitor comprises two contact terminals for the touching of the two hands or feet of a person; an amplifier-filter circuit adapted to sample impedance signal between the contact terminals, to amplify the impulse of the sampled impedance signal, and remove noises from the sampled impedance signal; a waveform converter adapted to rectify outputted waveform from the amplifier-filter circuit into a square wave; and a processing and output circuit adapted to receive and process the square wave signal outputted by the wave form converter, to obtain the mean frequency of peaks of the received square wave signal by means of a computing process, and to output the frequency value thus obtained to a display unit for display.

### BRIEF DESCRIPTION OF THE DRAWINGS

FIG. 1 is a heat beat curve obtained from a heat-rate monitor according to the prior art.

FIG. 2 is a circuit block diagram of a bi-point detection type heart-rate monitor according to the present invention.

FIG. 3 is a flow chart of the bi-point detection type heart-rate monitoring method according to the present invention.

FIG. 4 is a heat beat curve obtained from the bi-point detection type heart-rate monitor according to the present invention.

FIG. 5 is a schematic drawing showing the structure of an exercising machine in which the bi-point detection type heart-rate monitor of the present invention is installed.

FIG. 6 is a sectional view in an enlarged scale taken along line 6-6 of FIG. 5.

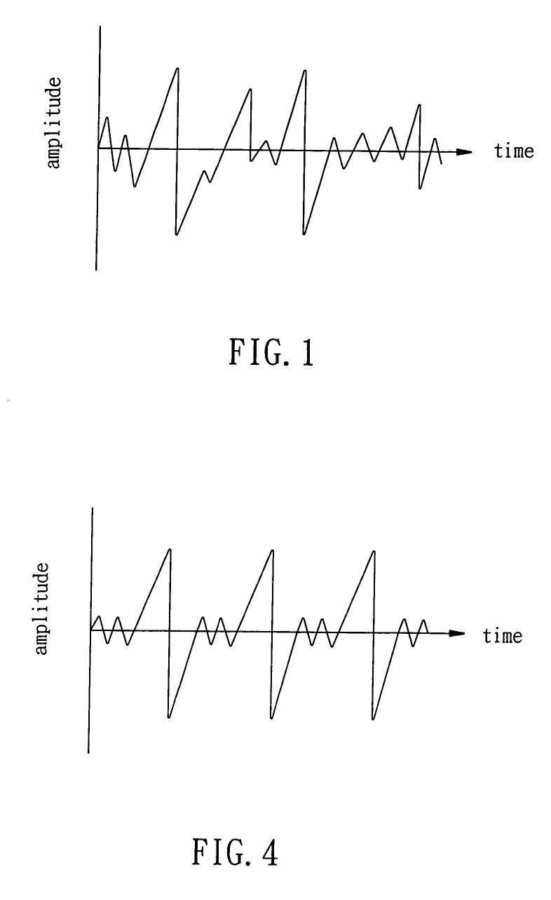

### DETAILED DESCRIPTION OF THE INVENTION

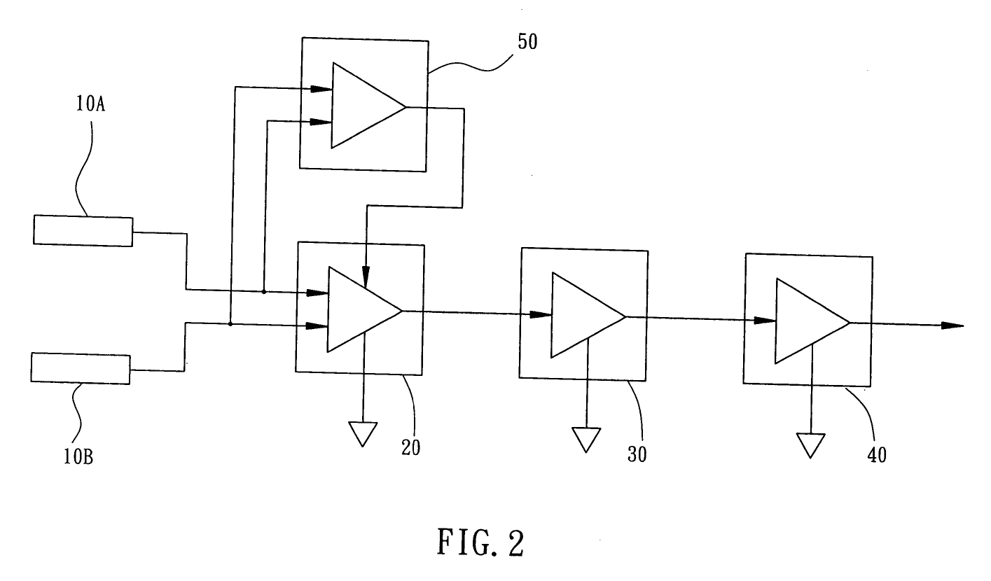

Referring to FIG. 2, a bi-point detection type heart-rate monitor in accordance with the present invention is generally comprised of two contact terminals 10A and 10B, an amplifier-filter circuit 20, a waveform converter 30, a processing and output circuit 40, and a detection unit 50.

The contact terminals 10A and 10B are electrically conducting terminals for the touching of the two hands or feet of the user (the person to be examined), i.e., the user works as a bioelectrical impedance connected between the contact terminals 10A and 10B when touching the contact terminals 10A and 10B accurately.

The amplifier-filter circuit 20 is adapted to apply a predetermined voltage to between the contact terminals 10A and 10B (enabling electric current to pass through the user's body), to sample impedance signal between the contact terminals 10A and 10B, and to amplify the impulse (i.e., the R wave) of the sampled impedance signal produced due to the beating of the user's heart and remove noises from the impulse.

The waveform converter 30 is adapted to rectify outputted waveform from the amplifier-filter circuit 20 into a square wave of predetermined wavelength.

The processing and output circuit 40 is a microprocessor adapted to receive the square wave signal outputted by the wave form converter 30, to obtain the mean frequency (indicative of the frequency of the beating heart) of the peaks of the received square wave signal by means of a computing process, and to output the frequency value thus obtained to a display unit (not shown) for display. The display unit can be an LCD, an LED display, or an LED matrix to display the frequency value by digits. Because the display unit is not within the scope of the claims of the present invention, no further detailed description in this regard is necessary.

The detection unit 50 is adapted to detect simultaneous touching of the contact terminals 10A and 10B by the user, and to let the processing and output circuit 40 output the aforesaid frequency value to the aforesaid display unit if the detection result is positive, or to terminate the detection action of the bi-point detection type heart-rate monitor and to drive the processing and output circuit 40 output a warning signal when the detection result is negative. In actual practice, the detection unit 50 can be comprised of two infrared inductors, thin-film switches, or micro switches respectively installed in the contact terminals 10A and 10B, and a specific circuit adapted to detect if the two sensors are simultaneously triggered or not and then to control the operation of the other circuit (for example, the amplifier-filter circuit 20) subject to the detection result.

The detection unit 50 is provided to prevent a false judgment and output of an error or insignificant value due to the interference of resistor means in the air with the circuit when the bi-point detection type heart-rate monitor is not in use or when the hands or legs of the person under examination unexpectedly left the contact terminals 10A and 10B during the use of the bi-point detection type heart-rate monitor.

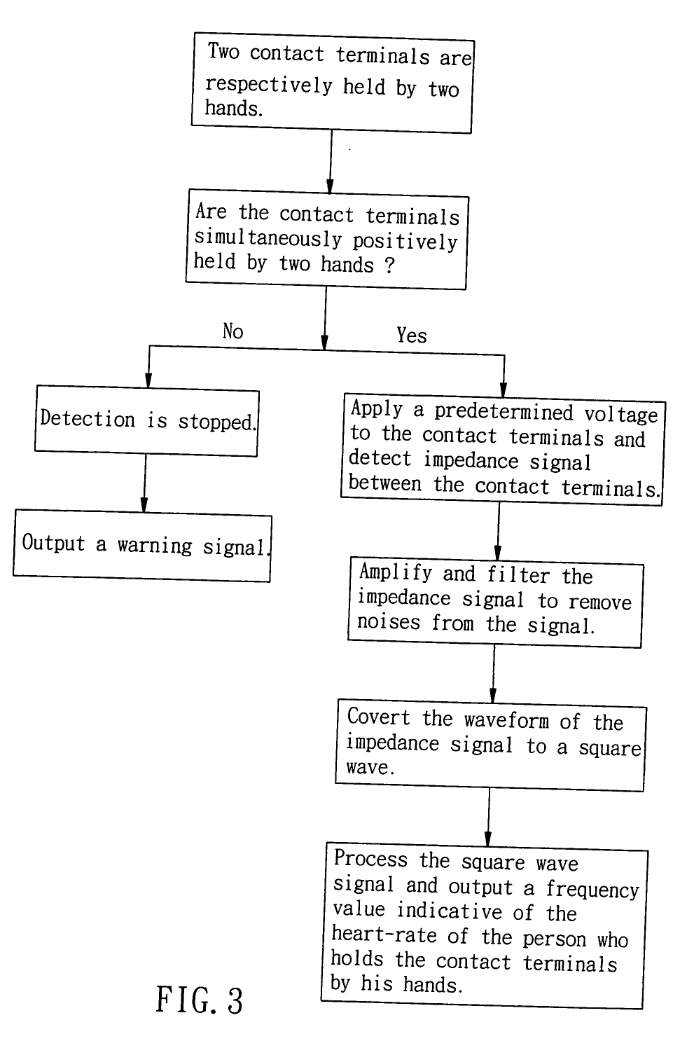

Referring to FIG. 3, the bi-point heart-rate monitoring method of the present invention comprises the steps of:
(a) letting the person to be examined to hold the two contact terminals 10A and 10B with the two hands;
(b) driving the detection unit 50 to detect if the two hands of the person to be examined are holding the two contact terminals 10A and 10B or not;
(c) stopping the detection and providing a warning signal if the detection of the detection unit 50 tells that the two hands of the person to be examined are not holding the two contact terminals 10A and 10B;
(d) applying a predetermined voltage to the two contact terminals 10A and 10B and simultaneously measure the impedance signal between the two contact terminals 10A and 10B if the detection of the detection unit 50 tells that the two hands of the person to be examined are holding the two contact terminals 10A and 10B; wherein since the detection unit 50 is provided, it prevents a false judgment of the circuit such that the impedance signal is as shown in FIG. 4;
(e) driving the amplifier-filter circuit 20 to amplify the impedance signal thus measured and to remove noises from the signal;
(f) driving the waveform converter 30 to rectify outputted waveform from the amplifier-filter circuit 20 into a square wave of a predetermined wavelength;
(g) driving the processing and output circuit 40 to receive the square wave signal outputted by the wave form converter 30 and to process the received square wave signal into a frequency value indicative of the heart-rate of the person under examination by means of a computing process, and then to output the frequency value thus obtained to a display unit for display.

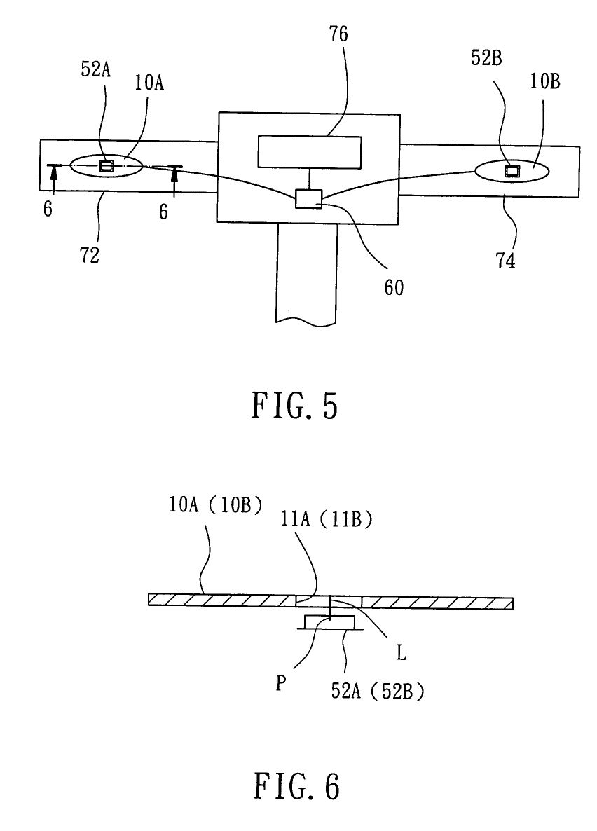

FIG. 5 is a schematic drawing showing the structure of an exercising machine in which the bi-point detection type heart-rate monitor of the present invention is installed. As illustrated, the first contact terminal 10A and the second contact terminal 10B are shaped like an oval plate and respectively located on the surfaces of the left handlebar 72 and right handlebar 74 of the exercising machine; the amplifier-filter circuit 20, the waveform converter 30, and the processing and output circuit 40 are integrated into a circuit board 60; two infrared transmitting receiving devices 52A and 52B (other equivalent sensor means may be used) are respectively installed in holes 11A and 11B in the oval plate-like first and second contact terminals 10A and 10B to form with a detection circuit in the circuit board 60 the aforesaid detection unit 50; the output end of the processing and output circuit 40 is electrically connected to a display unit 76. When the user holding the tow handlebars 72 and 74 over the contact terminals 10A and 10B during exercise, the bi-point detection type heart-rate monitor monitors the heart-rate of the user and displays the detected frequency value on the display unit 76.

With respect to further detailed description of the detection unit 50, please refer to FIG. 6. The infrared transmitting receiving devices 52A and 52B are respectively installed in the respective center holes 11A and 11B in the oval plate-like first and second contact terminals 10A and 10B, each having a transmitting terminal and a receiving terminal. The transmitting terminal and receiving terminal are located on the same location, which is referenced by P. When the user is holding the contact terminals 10A and 10B with the two hands, the infrared transmitting receiving devices 52A and 52B simultaneously produce a respective invisible light beam L that is not harmful to human beings. The transmitting terminal is the initial point of the respective light beam L. The receiving terminal is the receiving point of the diffraction light produced after the respective light beam L encountered an obstacle (the respective light beam L projected onto the palm of the corresponding hand of the user). By means of detecting energy variation at the receiving terminals of the infrared transmitting receiving devices 52A and 52B of the detection unit 50, the distance (corresponding to amount of loss of light energy) between the user (the person under examination) and the transmitting terminals is measured, and therefore the contact status between the palms of the user's hands and the contact terminals 10A and 10B is known. When the energy detected at the infrared transmitting receiving devices 52A and 52B of the detection unit 50 is within the set range, the monitor starts monitoring the user's heart-rate. When the energy detected at either of the infrared transmitting receiving devices 52A and 52B surpassed the set value, the monitor stops monitoring the user's heart-rate, and produces a warning signal. The monitoring action starts again only when the user adjusted to the accurate position.

As indicated above, the bi-point detection type heart-rate monitor has the following advantages:

1. The structural arrangement of the present invention is unlike conventional four-point detection type heart-rate monitors. When in use, each hand or foot needs only to touch one single terminal to achieve positive contact.

2. If either hand (or foot) of the user is not in contact with the respective terminal, the contact error is immediately detected without obtaining impedance from the air, and therefore the invention does not produce a false value, ensuring an accurate detection result.

3. The infrared transmitting receiving devices are respectively located on the geographical center of the respective contact terminals, increasing the detection accuracy of the system.

According to the present invention, the bi-point detection type heart-rate monitor can be employed to a variety of devices having footboard or pedal means (weight scales or pedals of exercising machines), devices having handle means (handlebars of exercising machines or physical measuring instruments), or any of a variety of other devices.

Although a particular embodiment of the invention has been described in detail for purposes of illustrated, various modifications and enhancements may be made without departing from the spirit and scope of the invention. Accordingly, the invention is not to be limited except as by the appended claims.

1. A bi-point detection type heart-rate monitor comprising:
two electrically conductive contact terminals for the touching of the two hands or feet of a person;
an amplifier-filter circuit adapted to sample impedance signal between said contact terminals, to amplify the impulse of the sampled impedance signal, and to remove noises from the sampled impedance signal;
a waveform converter adapted to rectify outputted waveform from said amplifier-filter circuit into a square wave; and
a processing and output circuit adapted to receive and process the square wave signal outputted by said wave form converter, to obtain the mean frequency of peaks of the received square wave signal by means of a computing process, and to output the frequency value thus obtained to a display unit for display.

2. The bi-point detection type heart-rate monitor as claimed in claim 1 further comprising a detection unit adapted to detect simultaneous touching of said contact terminals by the person to be examined, and to let said processing and output circuit output said frequency value to said display unit when the detection result is positive.

3. The bi-point detection type heart-rate monitor as claimed in claim 2, wherein said detection unit comprises two infrared transmitting receiving devices respectively installed in said contact terminals, and a detection circuit adapted to detect triggering of said infrared transmitting receiving devices.

4. The bi-point detection type heart-rate monitor as claimed in claim 3, wherein said infrared transmitting receiving devices are respectively located on the geographical center of said contact terminals.

5. The bi-point detection type heart-rate monitor as claimed in claim 4, wherein said contact terminals each have a hole disposed at the respective geographical center and adapted to accommodate said infrared transmitting receiving devices respectively.

6. The bi-point detection type heart-rate monitor as claimed in claim 2, wherein said detection unit comprises two thin-film switches respectively installed in said contact terminals, and a detection circuit adapted to detect trigging of said thin-film switches.

7. The bi-point detection type heart-rate monitor as claimed in claim 2, wherein said detection unit comprises two micro switches respectively installed in said contact terminals, and a detection circuit adapted to detect trigging of said micro switches.

8. The bi-point detection type heart-rate monitor as claimed in claim 1, wherein said processing and output circuit is a microprocessor.

9. A bi-point detection type heart-rate monitoring method comprising the steps of:
(a) letting the hands (legs) of the person to be examined hold a respective contact terminal;
(b) using a circuit to apply a predetermined voltage to the two contact terminals in contact the hands (legs) of the person to be examined, and then measuring the impedance signal between the two contact terminals;
(c) driving an amplifier-filter circuit to amplify the impedance signal thus measured and to remove noises from the signal;
(d) driving a waveform converter to rectify outputted waveform from the amplifier-filter circuit into a square wave;
(e) driving a processing and output circuit to receive and process the square wave signal outputted by said wave form converter, to obtain the mean frequency of peaks of the received square wave signal by means of a computing process, and to output the frequency value thus obtained to a display unit for display.

10. The bi-point detection type heart-rate monitoring method as claimed in claim 1 further comprising a sub-step of driving a detection unit to detect contact between the hands (legs) of the person to be examined and the respective contact terminals before step (a), and then proceeding to step (b) only when positive contact between the hands (legs) of the person to be examined and the respective contact terminals has been detected.

11. The bi-point detection type heart-rate monitoring method as claimed in claim 10, wherein said sub-step further stopping the monitoring action when said detection unit detected the hands (legs) of the person to be examined not simultaneously touched the respective contact terminals.

12. The bi-point detection type heart-rate monitoring method as claimed in claim 10, wherein said sub-step further producing a warning signal when said detection unit detected the hands (legs) of the person to be examined not simultaneously touched the respective contact terminals.
